(19) United States
(12) Patent Application Publication
(10) Pub. No.: US 2025/0049455 A1
(43) Pub. Date: Feb. 13, 2025

(54) SYSTEM AND METHOD FOR EFFICIENT INTRAVASCULAR LITHOTRIPSY

(71) Applicant: Vantis Vascular, Inc., Kalamazoo, MI (US)

(72) Inventors: Tim A. FISCHELL, Kalamazoo, MI (US); Michael S. MIRIZZI, San Jose, CA (US); Frank SALTIEL, Kalamazoo, MI (US); Jeffrey PAYNE, Temecula, CA (US)

(21) Appl. No.: 18/799,536

(22) Filed: Aug. 9, 2024

Related U.S. Application Data
(60) Provisional application No. 63/518,864, filed on Aug. 10, 2023.

(51) Int. Cl. A61B 17/22; A61M 25/00; A61M 25/01

(52) U.S. Cl. CPC A61B 17/22022; A61M 25/0105; A61B 2017/22025; A61B 2017/22062; A61M 2025/0006

(57) ABSTRACT

An intravascular lithotripsy apparatus is disclosed. The apparatus may include a balloon assembly that expands and provides shockwaves for any feasible lumen. The balloon assembly may be shielded by an outer catheter providing a smooth surface to ease advancement through the lumen. Also described herein are locking mechanism for securing dual catheters that may be incorporated with any of the apparatuses described.

### CLAIM OF PRIORITY

This patent application claims priority to U.S. Provisional Patent Application No. 63/518,864, titled “SYSTEM AND METHOD FOR EFFICIENT INTRAVASCULAR LITHOTRIPSY,” filed on Aug. 10, 2023, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods and apparatuses described herein may be related to intravascular lithotripsy procedures. More specifically, the methods and apparatuses described herein may relate to apparatuses that may enable a surgeon to efficiently and easily perform lithotripsy procedures.

### BACKGROUND

The treatment of coronary and peripheral arterial obstructive lesions has become a main focus of vascular therapy in the last many decades. Originally this was done with balloon angioplasty. Ultimately coronary and peripheral stents were also used to enhance the long-term revascularization of arterial blockage. One of the great limitations of both angioplasty and stenting relates to the inability to adequately expand devices in areas where there is vascular calcification.

A number of devices have been devised to treat calcium. This includes rotational and orbital atherectomy, and most recently the advent of intravascular lithotripsy (IVL). IVL procedures create shockwaves within a blood vessel or other lumen as a means to disrupt or “crack” the calcifications. The shockwaves are typically centered within a balloon catheter, that is typically expanded at the time of the intravascular lithotripsy to further enhance the effects of the shockwaves.

Conventional IVL treatments and procedures use bulky and stiff balloons and electrodes that can be extremely difficult to deliver down a diseased, calcified, and tortuous coronary arteries and/or peripheral arteries. In addition, because of the bulkiness and stiffness of the conventional IVL approaches, the balloon lengths are limited which makes treating longer segments of calcified vessels time consuming and inefficient.

### SUMMARY OF THE DISCLOSURE

Described herein are apparatuses, systems, and methods to deliver intravascular lithotripsy (IVL) to a patient. In some examples, the IVL therapies may be delivered through a balloon assembly that may be coupled to an inner catheter and selectively sheathed by an outer catheter. In this manner, the outer catheter can protect and/or shield the balloon assembly from exposure to the arteries or veins. This shielding eases insertion and advancement of the balloon assembly. In some cases, the shielding allows longer balloon assemblies compared to conventional IVL assemblies.

Any of the apparatuses described herein may include an inner catheter and an outer catheter. The inner catheter may include a tapered element and a balloon assembly. The tapered element may be a distal element disposed on a distal end of the inner catheter. The tapered element may be slender to enable easy insertion and advancement in the patient. The balloon assembly may be proximal to the tapered element. In general, the balloon assembly may include one or more pairs of electrodes. The electrodes may receive high voltage energy pulses with which to generate shockwaves by emitting electric arcs.

The outer catheter may include a lumen configured to concentrically surround the inner catheter and a tip, disposed on a distal end of the outer catheter. The tip is configured to slidably engage with a landing zone of the tapered element. In general, the tip may be made from any feasible material. In general, the material may be compliant and flexible and may seal to the tapered element (particularly when in contact of the landing zone of the tapered element). The material of the tip may resist fish-mouthing which may occur when the IVL assembly is advanced through curved blood vessels within the patient.

In any of the apparatuses described herein, the balloon assembly may be configured to expand or be inflated by the presence of a liquid or a gas. In general, the balloon assembly is expanded after the outer catheter has been retracted (with respect to the inner catheter) thereby exposing the balloon assembly.

In any of the apparatuses described herein, the balloon assembly may be configured to be contained within (e.g., covered, shielded, surrounded by, etc.) the outer catheter when the tip of the outer catheter is in contact with the landing zone of the tapered element.

Furthermore, in any of the apparatuses described herein, the outer catheter may further include an alignment window while the inner catheter further includes a midshaft marker configured to appear within the alignment window when the tip of the outer catheter is in contact with the landing zone of the tapered element. In this manner, the midshaft marker may provide positional information regarding the balloon assembly and the outer catheter while the balloon assembly is inserted into an artery or vein.

In any of the apparatuses described herein, the apparatus may include two or more markers configured to indicate a position of the balloon assembly with respect to the outer catheter when the intravascular lithotripsy apparatus is inserted into a patient. These markers may be associated with (marked on) the inner catheter or the outer catheter. In some variations, the two or more markers may remain outside of the patient, particularly when the balloon assembly is advanced within the patient.

As described herein, a control shaft (such as an outer catheter control shaft) may be coupled to a proximal end of the outer catheter. The control shaft may be used to control a position of the outer catheter. In some examples, the inner catheter may include a first set of marker bands configured to indicate when the balloon assembly is within the outer catheter. The marker bands may be compared to bands on the control shaft. In some other examples, the inner catheter may include a second set of marker bands configured to indicate when the balloon assembly is extended beyond the outer catheter.

Any of the apparatuses described herein may include a hub coupled to an inner catheter and a locking clip coupled to the control shaft. The locking clip may be used to lock the apparatus in a first or a second configuration. In the first configuration the balloon assembly is outside of the outer catheter and in the second configuration the balloon assembly is inside of the outer catheter.

In any of the apparatuses described herein, the two or more electrodes (within the balloon assembly) may be formed from a flex circuit, conductive ink, or a combination thereof Furthermore, in any of the examples, the tapered element may be about three centimeters long. In any of the apparatuses described herein, the tip of the outer catheter and the tapered element form a smooth outer surface when the tip of the outer catheter is slidably engaged with the landing zone of the tapered element.

Any of the apparatuses described herein may support removal of the inner catheter. For example, the inner catheter may be configured to be removed (withdrawn) from the outer catheter. In some variations, the balloon assembly may be between two and eight millimeters in diameter when expanded or inflated. In some other variations, the balloon assembly may be at least 20 millimeters long.

A method for treating a patient with shockwaves is described herein. In general, the method may use any of the apparatuses described herein, such as an IVL system that includes an inner catheter, an outer catheter and a balloon assembly. The method may include percutaneously inserting an intravascular lithotripsy apparatus into a blood vessel of a patient, wherein the intravascular lithotripsy apparatus includes a balloon assembly coupled to an inner catheter and an outer catheter configured to surround the balloon assembly, advancing the intravascular lithotripsy apparatus to a treatment region, retracting the outer catheter to expose the balloon assembly, and inflating the balloon assembly and delivering shockwaves to the treatment region.

In any of the methods described herein, advancing the intravascular lithotripsy apparatus may include locking the inner catheter and the outer catheter together. The catheters may be locked together using any feasible locking mechanism including those described herein. In addition, any of the methods described herein may include unlocking the inner catheter from the outer catheter; and moving the outer catheter distally based on one or more marker rings disposed on the inner and outer catheters. In some examples, retracting the outer catheter may include viewing a marker ring with respect to a transparent alignment window disposed on the outer catheter.

Any of the methods described herein may include deflating the balloon assembly and withdrawing the inner catheter from the outer catheter.

Locking mechanisms are described herein that temporarily lock or bind together two or more catheters or shafts. Locking or binding the shafts together may keep the balloon assembly either shielded by the outer catheter or keep the balloon assembly exposed with respect to the outer catheter.

In general, the locking mechanisms described herein may be referred to as locks, or proximal locks. These locks may be configured to be adjustably positioned over and around the two or more shafts. In some cases these locks may be placed over the shafts without requiring sliding along the shafts (e.g., clamped over the shafts). In some examples the locks may hold the shafts, which may be of different diameters, in tandem. The lock may include separate channels for securing each of the shafts. The channels may be of different sizes to accommodate differently sized shafts. The channels may be configured to grip or retain the shafts. In some cases the channels may be lined or may include a textured material and/or a material having a gripping surface (e.g., a high tack surface). In some cases the channels may include a compressible material (e.g., open or closed cell foam, etc.).

A locking mechanism for temporarily binding together two or more shafts may include a cylindrical body configured to receive two or more shafts through a central opening, a compliant grommet configured to bind the two or more shafts together in response to a compression force, and a plunger configured to provide a variable compression force to the compliant grommet.

In any of the locking mechanisms described herein, the compliant grommet may be configured to receive the two or more shafts through a central opening concentric with the cylindrical body. In some examples, the plunger may include a button configured to lock the plunger in a position away from the compliant grommet. Any of the locking mechanisms described herein may include a spring configured to provide a bias force against the plunger. In some variations, a spring may be configured to provide a bias force against the plunger. In some other variations, the plunger may be configured to provide the compression force based at least in part on the bias force. In any of the locking mechanisms described herein, the body may include a slot configured to hold the plunger away from the grommet.

In some examples the locking mechanism may include a clamp and a body. The clamp may include locating pins, a first guide tube, a second guide tube and an arm coupling the first guide tube to the second guide tube, wherein the first guide tube and the second guide tube are configured to receive the two or more shafts. The body may be flexibly coupled to the clamp and include slots to movably couple to the pins.

In any of the locking mechanisms described herein, the arm may be further configured to position the first guide tube at an angle with respect to the second guide tube and bind together the two or more shafts.

In any of the locking mechanisms described herein, the arm is further configured to receive a deflection force and reduce an angle between the first guide tube and the second guide tube and enable the two or more shafts to slide freely within the first guide tube and the second guide tube. In some examples, the first guide tube and the second guide tube include a slot configured to receive the two or more shafts.

Yet another locking mechanism is described. The locking mechanism may include a cam and a body. The body may include a slot configured to receive the two or more shafts, and an opening configured to receive the cam, wherein the cam is configured to contact and bind the two or more shafts to the body. The cam can include a handle and may be configured to rotate one-fourth of a whole turn to bind the two or more shafts to the body. In any of the locking mechanisms described herein, the cam may include an elastomeric stopper to contact the shafts.

In any of the locking mechanisms described herein, the elastomeric stopper is at least one of rubber, thermoplastic urethane, or silicon. Furthermore, in any of the locking mechanisms described herein, the cam may be configured to allow the shafts to move freely in a first position and to bind the shafts together in a second position.

Still another locking mechanism is described. The locking mechanism may include a locking clip coupled to a first shaft and a locking ring coupled to a second shaft and including a groove configured to receive the locking clip. In any of the locking mechanisms described herein, the locking clip may be laser welded to the second shaft. In other examples, the locking clip is nitinol.

In any of the locking mechanisms described herein, the locking clip may be formed by the second shaft. In some examples, the locking ring is at least one of a machined, welded, or insert molded material. In some other examples, the second shaft is formed from nitinol.

Another locking mechanism is disclosed. The locking mechanism may include a notch feature formed in a first catheter shaft, and a snap ring coupled to a second catheter shaft and configured to receive the notch feature. In any of the locking mechanisms described herein, the first catheter shaft is formed from nitinol.

A locking mechanism is described. The locking mechanism may include a clam shell body and an elastomeric pad. The clam shell body may include a first member, a second member, and a hinge member configured to movably couple the first member to the second member. The elastomeric pad may be configured to fit within the first member and bind together two or more shafts.

In any of the locking mechanisms described herein, the second member can include at least one groove configured to receive at least one shaft. In some examples, the clam shell body can be injection molded.

In any of the locking mechanisms described herein, the clam shell body may include a latch. The latch may include a ramp member molded onto an edge of the first member, the edge of the first member disposed across from the hinge member, and a beam member molded onto an edge of the second member, the edge of the second member disposed across from the hinge member, the beam member configured to engage and detachably couple to the ramp member. In some examples, the ramp member is configured to deflect the beam member as the first member is rotated to the second member. Furthermore, in some cases the beam member may be configured to engage a locking surface of the ramp member.

In any of the locking mechanisms described herein, the elastomeric pad is at least one of thermoplastic urethane or silicon.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a simplified block diagram of an example intravascular lithotripsy (IVL) system.

FIG. 2 is a block diagram showing some example components of the generator of FIG. 1.

FIG. 3A is an example view of a distal portion of a catheter assembly.

FIG. 3B shows a simplified block diagram of a catheter assembly configured to deliver shockwave treatment.

FIGS. 4A and 4B illustrate different modes of the catheter assembly of FIG. 3.

FIGS. 5A-5C show details regarding controlling the alignment between catheters of a catheter assembly, such as the catheter assembly of FIG. 3.

FIGS. 6A and 6B show detailed views of an inner catheter and an outer catheter control shaft.

FIG. 7A shows a first example view of an inner catheter and an outer catheter control shaft.

FIG. 7B shows a detailed view of the inner catheter and the outer catheter in a first configuration.

FIG. 7C shows a second example view of the catheter and the outer catheter control shaft.

FIG. 7D shows a detailed view of the inner catheter and the outer catheter in the second configuration.

FIGS. 8A-8C show various components of a catheter assembly disposed in a first configuration.

FIGS. 9A-9C show various components of a catheter assembly disposed in a second configuration.

FIGS. 10A and 10B show various views of a hub that may be used in conjunction with the catheter assembly of FIG. 3.

FIGS. 11A and 11B show an example implementation of a latch mechanism.

FIGS. 12A-12F show a first midshaft locking mechanism.

FIGS. 13A and 13B show a second midshaft locking mechanism.

FIGS. 14A and 14B show a third midshaft locking mechanism.

FIGS. 15A-15C show a fourth midshaft locking mechanism.

FIGS. 16A and 16B show a fifth midshaft locking mechanism.

FIGS. 17A and 17B show a sixth midshaft locking mechanism.

FIGS. 18A and 18B show a seventh midshaft locking mechanism.

FIGS. 19A-19E show an eighth midshaft locking mechanism.

FIG. 20 is a flowchart showing an example method for treating a patient with an IVL system.

### DETAILED DESCRIPTION

In general, intravascular lithotripsy methods can be used to treat calcified arteries or other lumens. Previously, the length and/or diameter of the lithotripsy balloons used to deliver the shockwave has been limited to permit insertion and placement through clogged or twisting arteries.

Described herein is a intravascular lithotripsy system that can use lithotripsy balloons that are advantageously longer than conventional lithotripsy balloons. The increased length (and sometimes increased diameter) may be feasible by a unique arrangement of catheters that may be used to insert and position the lithotripsy balloons at a treatment site or region. In some examples, the lithotripsy balloon may be sheathed by an outer catheter so that the balloon may slide by any difficult areas in transit to the treatment region. At the treatment region, the outer catheter may be retracted (or the inner catheter may be extended) to expose and deploy the lithotripsy balloon. In addition, described herein are multiple ways to bind together two or more catheters or shafts that may be used during lithotripsy treatment.

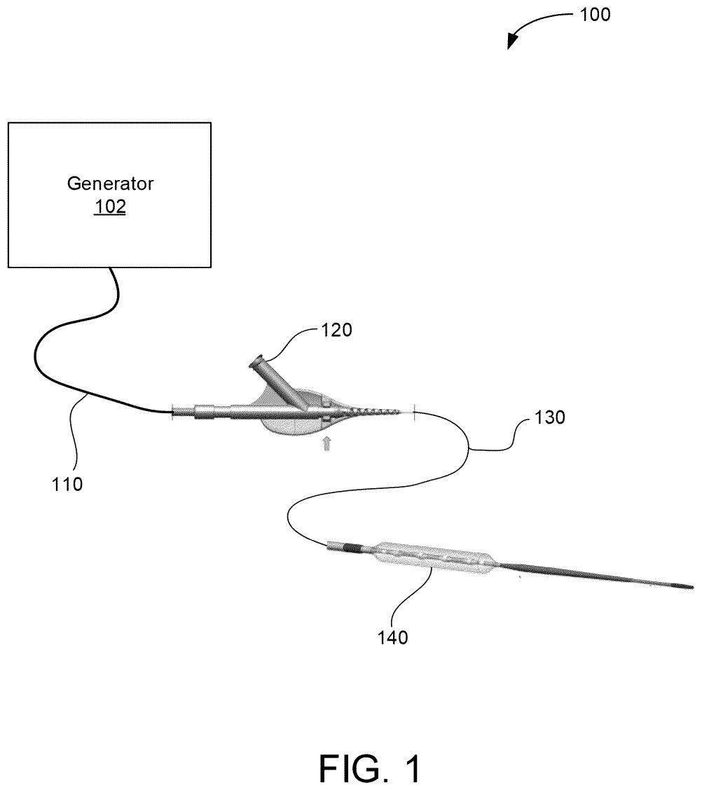

FIG. 1 is a simplified block diagram of an example intravascular lithotripsy (IVL) system 100. The IVL system 100 may include a generator 102, a cable 110, a catheter hub 120, a catheter assembly 130, and a balloon assembly 140. The generator 102 may include systems and devices to control one or more aspects of the IVL system 100, including operations of the balloon assembly 140. For example, the generator 102 may provide energy (voltage and/or current) to the balloon assembly 140. The energy may be used to induce one or more arcs between electrodes enclosed in the balloon assembly 140. The arcs may deliver shockwaves (pressure waves) to a treatment area or region. The energy may be provided to the balloon assembly 140 from the generator 102 through a removably coupled cable 110. The cable 110 may be coupled to the catheter hub 120 which may be coupled through one or more catheters (included in the catheter assembly 130) to the balloon assembly 140.

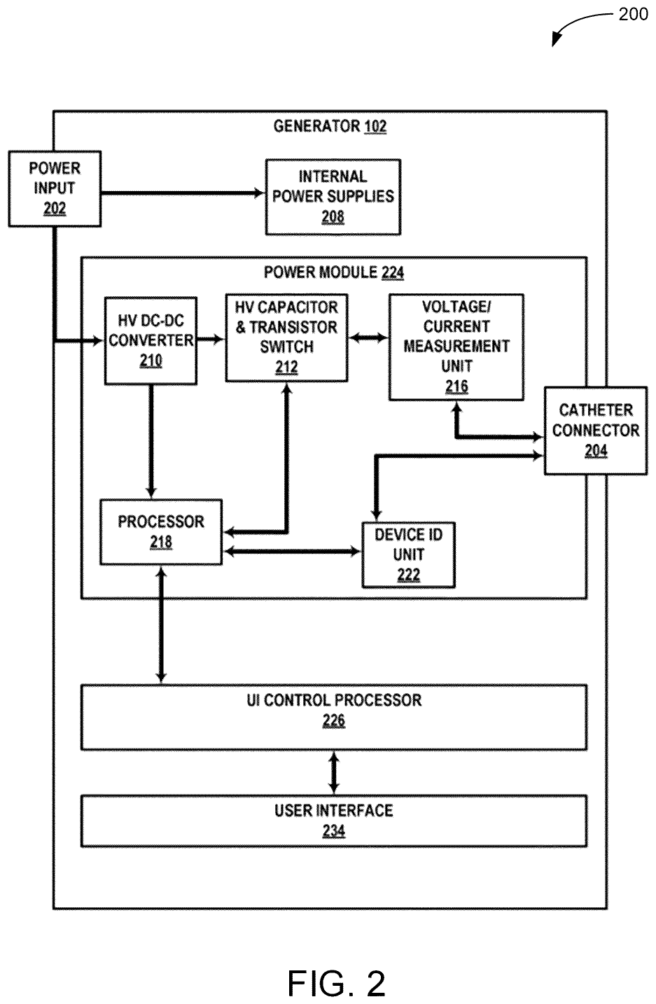

Any fluids for inflating the balloon assembly 140 can be administered through the catheter hub 120. In addition, the catheter hub 120 may enable a clinician (physician or other trained personnel) to control and introduce the balloon assembly 140 and the catheter assembly 130 percutaneously into a patient. The catheter hub 120, the catheter assembly 130 and the balloon assembly is discussed in more detail below with respect to FIGS. 2-11.

FIG. 2 is a block diagram 200 showing some example components of the generator 102 of FIG. 1. A power input 202 (e.g., for conductively coupling to a wall port or another electricity source) connects to power module 224 and an internal power supply 208. As shown in FIG. 2, power module 224 can include a high-voltage DC-DC converter 210, a high-voltage capacitor and transistor switch 212, a voltage and/or current measurement unit 216, and a device identification unit 222, configured to determine whether a coupled catheter is an authorized device while the catheter is connected via a catheter connector 204. For instance, the generator 102 may be configured to disable energy output to catheter connector 204 when an unidentified device is connected. In some examples, the catheter connector 204 may be coupled to a cable (such as cable 110 of FIG. 1) and to a balloon assembly (such as the balloon assembly 140). In this manner, the generator can provide energy to the balloon assembly 140 to induce electrical arcs.

The generator 102 can include a memory and one or more processors, such as processor 218 and/or user-interface-control processor 226. UI control processor 226 is configured to provide functionality for the user interface 234 of energy generator 102, such as a display screen, touch screen, buttons, or other manual controls enabling a user (e.g., a clinician) to operate the generator 102.

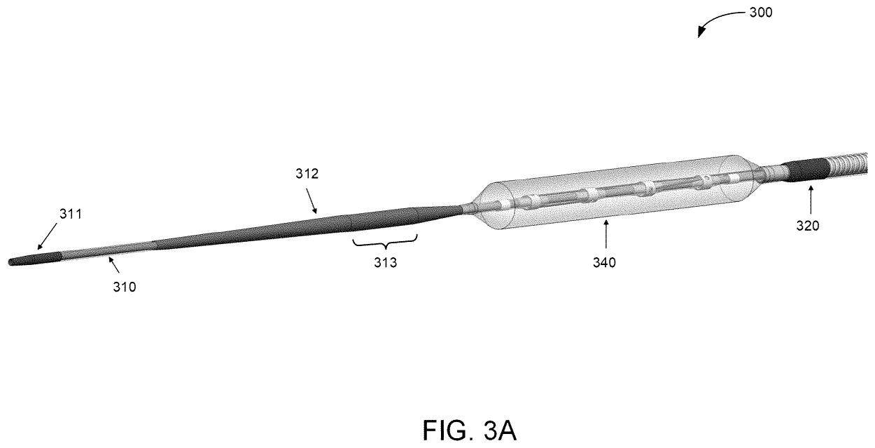

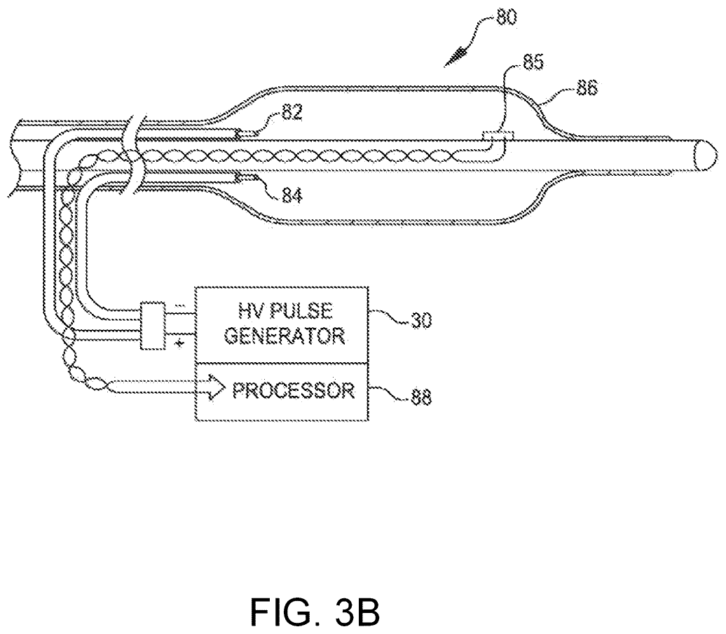

FIG. 3A is an example view of a distal portion of a catheter assembly 300. In some examples, the catheter assembly 300 may include the balloon assembly 140 and at least a portion of the catheter assembly 130, both of FIG. 1. The catheter assembly 300 may include an inner catheter 310 and an outer catheter 320. In addition, a balloon assembly 340 may be coupled to the inner catheter 310. The inner catheter 310 may be slidably coupled to the outer catheter 320. The outer catheter 320 may be a lumen configured to concentrically surround and slidably couple to the inner catheter. In some examples, the inner catheter 310 may fit within, and be encircled by, the outer catheter 320. In some embodiments, the inner catheter 310 may be formed from or include any lubricious material, including materials that include polytetrafluoroethylene (PTFE) components.

The balloon assembly 340 is shown in FIG. 3A in an expanded (deployed) configuration. The catheter assembly 300 may be advanced toward a treatment region of the patient as the inner catheter 310 and the outer catheter 320 are guided into the patient. The inner catheter 310 may include a tapered tip 311, a tapered element 312, and a landing zone 313. Some or all of the inner catheter 310 may include a flexible coiled shaft. Distally located furthest away from the catheter hub (not shown) is the tapered tip 311. The tapered tip 311 may be soft and a traumatically shaped. In some variations, the tapered tip 311 may be radiopaque to enable easy and accurate tracking within the patient during treatment. The tapered element 312, which can lie between the tapered tip 311 and the landing zone 313 may be part of the inner catheter 310 that transitions from a relatively narrow tapered tip to a relatively wider landing zone 313. In some cases, the tapered element 312 can enable the inner catheter 310 to be inserted and guided past restrictions or obstacles within a blood vessel or other lumen.

The outer catheter 320 may include a tip 321 distally located on the outer catheter 320. The tip 321 may be soft and radiopaque. When the balloon assembly 340 is not deployed, the balloon assembly 340 is contained within the outer catheter 320. For example, the outer catheter 320 may be advanced with respect to the inner catheter 310 until the tip 321 is coincident with and is in contact with the landing zone 313. In some variations, the tip 321 and the landing zone 313 may be sized for an interference fit. The tip 321 may be formed of a conforming and/or elastomeric material to resist “fishmouthing” as the inner catheter 310 and the outer catheter 320 are advanced within the patient.

FIG. 3B shows a simplified block diagram of a catheter assembly 80 configured to deliver shockwave treatment. The catheter assembly may include a balloon assembly 86 which may be another example of the balloon assembly 340. Electrodes 82 and 84 may be coupled to a generator, such as a high voltage pulse generator 30. The high voltage pulse generator 30 may be wholly or partially included within the generator 102 of FIGS. 1 and 2.

The catheter assembly 80 may further include a sensor 85 to detect reflected signals. Reflected signals from the calcified plaque can be processed by a processor 88 to determine quality of the calcification and quality of pulverization of any lesion.

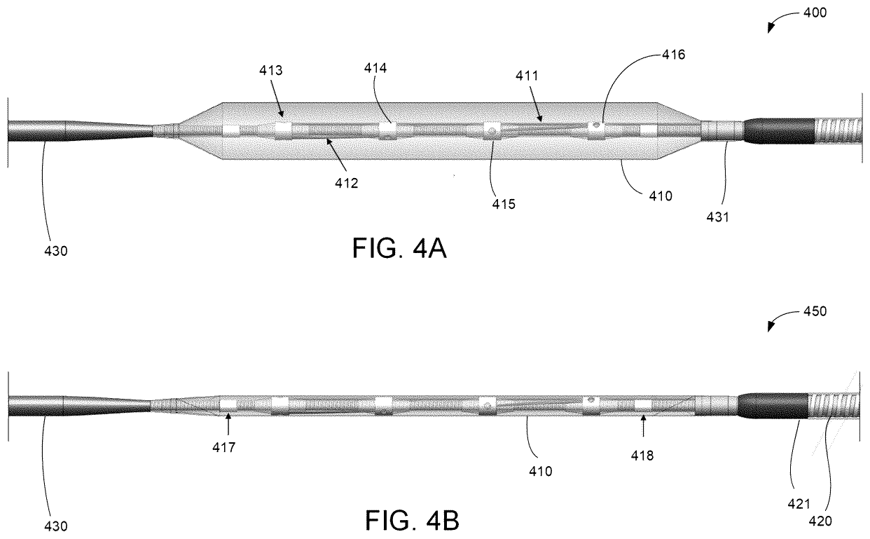

FIGS. 4A and 4B illustrate different modes of the catheter assembly of FIG. 3. In particular, FIG. 4A shows a first mode of an example catheter assembly 400. Similar to FIG. 3, the catheter assembly 400 can include a balloon assembly 410, an outer catheter 420, and an inner catheter 431. When operating in the first mode, the balloon assembly 410 can be expanded. In some variations, a fluid may be delivered through a catheter to fill and expand the balloon assembly 410.

The balloon assembly 410 can include one or more electrical conductors and one or more IVL electrodes. For example, a balloon of the balloon assembly 410 may include or surround a first wire 411 and a second wire 412. The first and second wires 411 and 412 may deliver high voltage to one or more coupled electrodes. In the example catheter assembly 400, the first and second wires 411 and 412 may be coupled to IVL electrodes 413-416. Each of the IVL electrodes 413-416 may include separate conductors (not shown) that may be coupled to the first and second wires 411 and 412. Although four IVL electrodes 413-416 are shown here, in other embodiments, the balloon assembly 410 can include any feasible number of electrodes. In some examples, the balloon assembly 410 may be approximately 20 millimeters long, 30 millimeters long, or any other feasible length. Furthermore, the balloon assembly 410 may have an expanded (inflated) diameter of between two and eight millimeters, although other diameters are contemplated.

As a high voltage signal is coupled to the first wire 411 and the second wire 412 (for example, from the generator 102 of FIG. 1), an electrical arc may be emitted from the IVL electrodes 413-416. The electrical arc may cause a pressure wave to be transmitted from the IVL electrodes 413-416 through the balloon assembly 410. When the balloon assembly 410 has been expanded so that the balloon is in contact with blood vessel or other lumen walls, a related pressure wave may be transferred circumferentially through the balloon to the blood vessel or other lumen walls. In this manner, a pressure wave may impinge and otherwise affect areas of calcification in a lumen. In some cases, the pressure waves may enable better patient outcomes associated with subsequent angioplasty and/or stent procedures.

FIG. 4B shows a second mode of an example catheter assembly 450. When operating in the second mode, the balloon assembly 410 can be collapsed, as shown. In the second mode, the balloon assembly 410 may easily fit within the outer catheter 420. The balloon assembly 410 may include a distal marker band 417 and a proximal marker band 418. In some variations, the distal marker band 417 and the proximal marker band 418 may be radiopaque to enable easy and rapid determination and confirmation of the location of the balloon assembly 410, particularly when the balloon assembly is inserted within the patient.

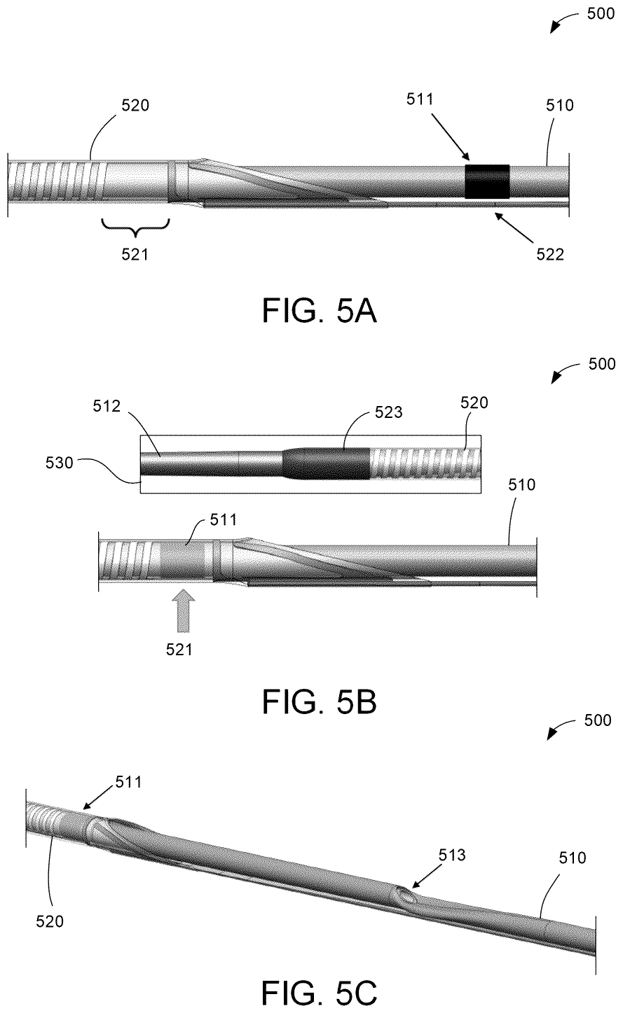

FIGS. 5A-5C show details regarding controlling the alignment between catheters of a catheter assembly, such as the catheter assembly 300 of FIG. 3. As described above, a portion of the outer catheter can be positioned on the inner catheter such that the distal tip of the outer catheter engages with the landing zone of the inner catheter. This configuration may enable smooth and easy insertion into and through blood vessels or other lumens of the patient.

FIG. 5A shows a portion of a catheter assembly 500 that includes an inner catheter 510 and an outer catheter 520. The inner catheter 510 may include a midshaft marker 511 while the outer catheter 520 may include an alignment window 521 and an outer catheter control shaft 522. The inner catheter 510 may be inserted and advanced into the outer catheter 520. In some cases, the inner catheter 510 is inserted into the outer catheter 520 before the catheter assembly 500 is inserted into the patient. In some variations, the junction between the inner catheter 510 and the outer catheter 520 as shown in FIG. 5A may be outside of the patient, even when the catheter assembly 500 in advanced into a treatment position.

To assist the clinician in obtaining and/or determining proper alignment between the inner catheter 510 and the outer catheter 520, the clinician may use the midshaft marker 511 and the alignment window 521. The alignment window 521, which may be transparent, may show the midshaft marker 511 when proper alignment between the inner catheter 510 and the outer catheter 520 such that the distal tip of the outer catheter 520 is engaged with the landing zone of a distal tip of the inner catheter 510.

FIG. 5B shows the portion of the catheter assembly 500 of FIG. 5A when the inner catheter 510 has been inserted into the outer catheter 520 such that a distal tip 523 of the outer catheter 520 is engaged or otherwise within the landing zone of the inner catheter 510. As shown, the inner catheter 510 can be advanced so that the midshaft marker 511 appears inside the alignment window 521. FIG. 5B also shows a detailed view of the distal tip 523 of the outer catheter 520. In some variations, when the midshaft marker 511 is within the alignment window 521, then the distal tip 523 may be placed in the landing zone of the tapered element 512 of the inner catheter 510 as shown in detail 530. In some variations, the midshaft marker 511 may be radiopaque to enable a more precise determination of the relationship between the inner catheter 510 and the outer catheter 520.

FIG. 5C shows another view of the portion of the catheter assembly 500 of FIG. 5B. The inner catheter 510 is inserted into the outer catheter 520 such that the midshaft marker 511 is within the alignment window 521. Also shown is a guide wire port 513 that can receive a guide wire or other implement.

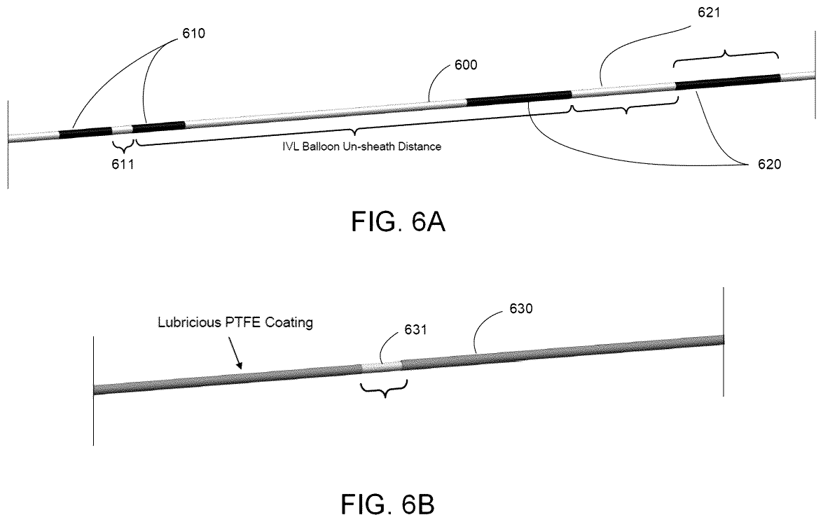

FIGS. 6A and 6B show detailed views of an inner catheter 600 and an outer catheter control shaft 630. As described above, the outer catheter control shaft 630 may be coupled to an outer catheter. Both the inner catheter 600 and the outer catheter control shaft 630 may include markers that provide positional feedback associated with a balloon assembly to the clinician. For example, FIG. 6A shows markings on the inner catheter 600. The inner catheter 600 may include two distal bands 610 and two proximal bands 620. In some variations, the outer catheter control shaft 630 may include any number of distal and/or proximal bands.

The distal bands 610 may be applied to a first position of the inner catheter 600. The distal bands 610 may be laser marked, pad printed, or heat shrink-based bands. The distal bands 610 may be approximately 5 mm wide. In some variations, the distal bands 610 can be any feasible width. Between the distal bands 610 may be a region 611. That separates the distal bands 610. In some implementations, the region 611 can be approximately two millimeters wide. In other examples, the region 611 can be any feasible width. The distal bands 610 may be used to indicate when the inner catheter 600 is in a first position with respect to the outer catheter. In some examples, the first position may be a position where the distal tip of the outer catheter is positioned on the landing zone of a tapered element of the inner catheter 600 as shown in FIG. 5B.

The inner catheter 600 may also include proximal bands 620. In some examples, the proximal bands 620 may be between approximately ten millimeters long wide. In some other examples, the proximal bands 620 can be any feasible width. A region 621 can separate the proximal bands 620. In some examples, the region 621 may be approximately ten millimeters wide, however any other feasible width is contemplated. The proximal bands 620 may be used to indicate when the inner catheter 600 is in a second position with respect to the outer catheter. In some examples, the second position may be a position where the distal tip of the outer catheter is positioned off of the landing zone and the balloon assembly is exposed and/or expanded. For example, in the second position, the distal tip 421 may be moved from the landing zone 430 allowing the balloon assembly 410 on the landing zone 430 of a tapered element as shown in FIG. 4A. The distal bands 610 and the proximal bands 620 may be separated by a balloon unsheathing distance. That is, the distal bands 610 may be separated from the proximal bands 620 by a distance associated with exposing or unsheathing the balloon assembly 410. Notably, the distal bands 610 and the proximal bands 620 may remain outside of the patient while the patient is undergoing treatment.

FIG. 6B shows a detailed view of the outer catheter control shaft 630. The outer catheter control shaft 630 may include a marking band 631. The marking band 631 may be laser ablated, masked, pad printed, or marked or formed with any feasible method or operation. In some examples, the marking band 631 can be approximately two millimeters wide. The marking band 631 can be used in conjunction with the distal bands 610 and/or the proximal bands 620 to enable the clinician to determine and control the relative positions of the outer catheter and the inner catheter 600. The outer catheter control shaft 630 may be coated with a lubricious coating, including but not limited to polytetrafluoroethylene (PTFE).

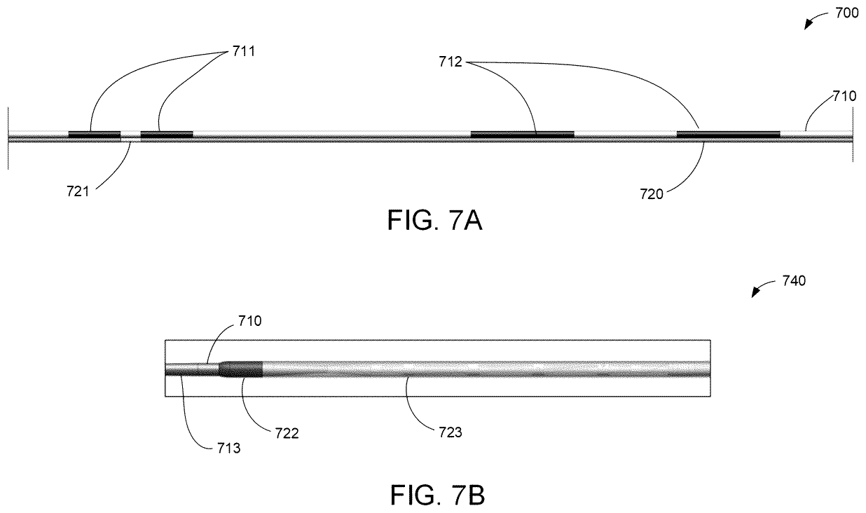

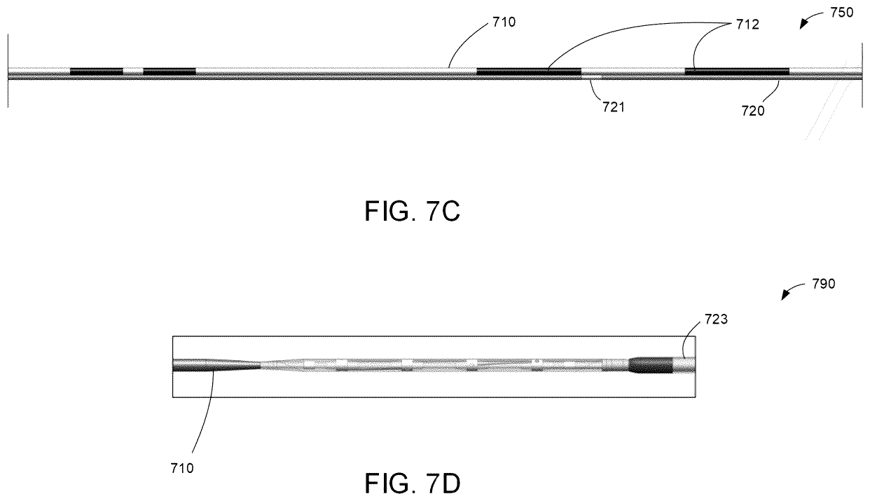

FIG. 7A shows a first example view 700 of an inner catheter 710 and an outer catheter control shaft 720. The inner catheter 710, which can be an example of the inner catheter 600 of FIG. 6, can include distal bands 711 and proximal bands 712. The outer catheter control shaft 720, which can be an example of the outer catheter control shaft 630, can include a marking band 721.

As described above, the marking band 721 in conjunction with the distal bands 711 and the proximal bands 712 can provide positional information to the clinician. As shown, the marking band 721 can lie approximately between the distal bands 711 thereby placing the catheter assembly in a first position or configuration. For example, when the marking band 721 is disposed approximately between the distal bands 711, then the distal tip of the outer catheter may be disposed approximately on the landing zone of a tapered tip of the inner catheter 710.

FIG. 7B shows a detailed view 740 of the inner catheter 710 and the outer catheter 723 in a first configuration. The outer catheter 723, which may be coupled to an outer catheter control shaft (not shown in FIG. 7B), may be positioned such that the marking band 721 is between the distal bands 711. In the first configuration, a distal tip 722 of the outer catheter 723 may be positioned on a landing zone of a tapered tip 713 of the inner catheter 710. Thus, in this first configuration, the catheter assembly may be configured for smooth insertion into a position for a patient.

FIG. 7C shows a second example view 750 of the inner catheter 710 and the outer catheter control shaft 720. The inner catheter 710 and the outer catheter control shaft 720 is shown in a second position or configuration. For example, the marking band 721 may be positioned between the proximal bands 712. When the outer catheter control shaft 720 is in this positional relationship with the inner catheter 710, then the outer catheter may be positioned to expose a balloon assembly.

For example, FIG. 7D shows a detailed view 790 of the inner catheter 710 and the outer catheter 723 in the second configuration. In the second configuration, the outer catheter 723 may no longer be disposed over the landing zone, but instead may be disposed proximally with respect to the landing zone. In some examples, a proximal position of the distal tip of the outer catheter may allow the balloon assembly to be exposed or extruded from the outer catheter.

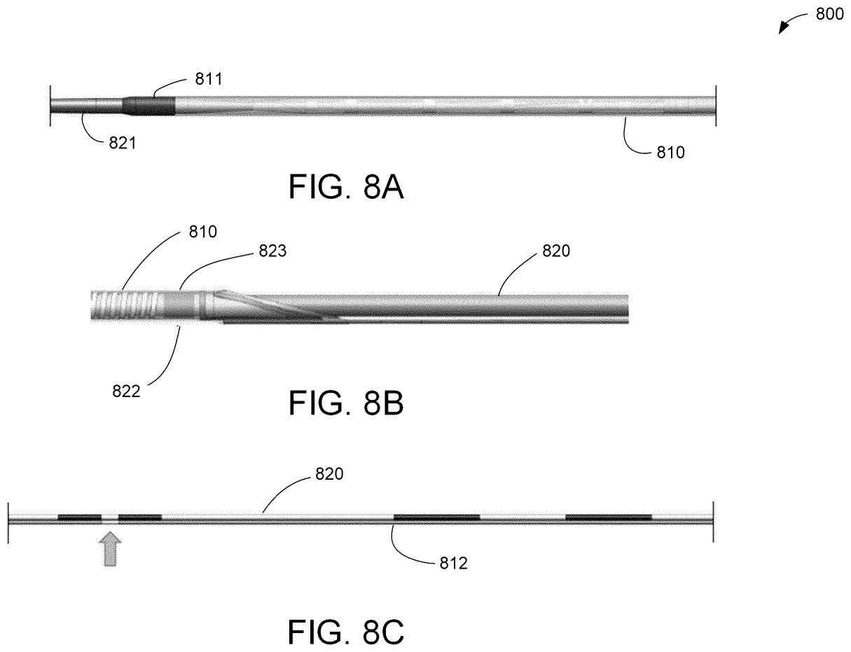

FIGS. 8A-8C shows various components of a catheter assembly 800 disposed in a first configuration. The first configuration may position a balloon assembly coupled to the inner catheter completely within an outer catheter. As shown in FIG. 8A, in the first configuration, an outer catheter 810 may be positioned on a landing zone of a tapered element 821 coupled to an inner catheter. In such a configuration, a distal tip 811 of the outer catheter may be positioned to engage with the tapered element 821 of the inner catheter. The inner catheter may be an example of the inner catheter 510 of FIG. 5A and the outer catheter 810 may be an example of the outer catheter 520 of FIG. 5A.

As shown in FIG. 8B, a midshaft marking band 822 of the inner catheter 820 may be positioned within a window of the outer catheter 810. When the midshaft marking band is disposed within the window 823, then the relationship of the inner catheter 820 and the outer catheter 810 may be such that the balloon assembly is covered or sheathed by the outer catheter 810.

FIG. 8C shows an inner catheter 820 and an outer catheter control shaft 812 coupled to the outer catheter (not shown). In the first configuration, the outer catheter control shaft 812 may be positioned such that a marking band of the outer catheter control shaft 812 is approximately between distal bands of the inner catheter. When the inner catheter and the outer catheter control shaft are disposed as shown in FIG. 8C, then the associated catheter assembly may be in a first configuration such that the balloon assembly is sheathed by the outer catheter.

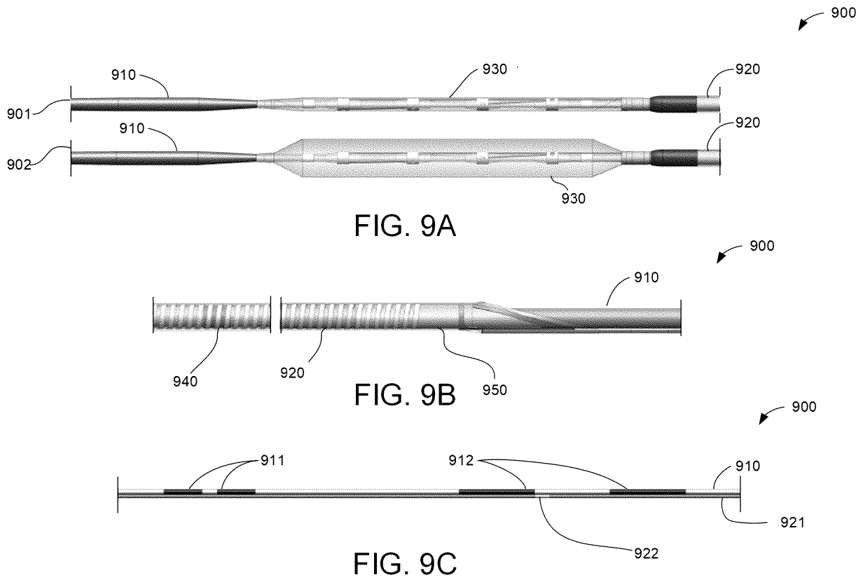

FIGS. 9A-9C show various components of a catheter assembly 900 disposed in a second configuration. The catheter assembly 900 may include an inner catheter 910, an outer catheter 920, and a balloon assembly 930. In some examples, the inner catheter 910 may be an example of the inner catheter 310 and the outer catheter 920 may be an example of the outer catheter 320, both from FIG. 3. In addition, the balloon assembly 930 can be an example of the balloon assembly 410 of FIG. 4.

As described above, the second configuration may position the balloon assembly 930 coupled to the inner catheter 910 beyond and/or outside of the outer catheter 920. As shown in FIG. 9A, in the second configuration, the balloon assembly 930, which is coupled to the inner catheter 910, is exposed or un-sheathed from the outer catheter 920. In view 901, the balloon assembly 930 is shown un-expanded or collapsed. In view 902, the balloon assembly 930 is shown expanded. The balloon assembly 930 may be inflated or expanded by an application of a liquid or gas. In some examples, the balloon assembly 930 may have an expanded diameter of between two and eight millimeters. In addition, the balloon assembly 930 may have a length between 20 and 30 millimeters long.

FIG. 9B shows a detailed view of the catheter assembly 900 showing the inner catheter 910 and the outer catheter 920. In the second configuration, the inner catheter 910 may be positioned with respect to the outer catheter 920 such that a midshaft marking band 940 is beyond (e.g., positioned distally) with respect to a window 950 on the outer catheter 920. The midshaft marking band 940 may be an example of the midshaft marking band of FIG. 8. As described in FIG. 8B, when the midshaft marking band 940 is within the window 950, then the balloon assembly (not shown) may be within the outer catheter 920. On the other hand, when the midshaft marking band 940 is positioned distally with respect to the window 950, then the balloon assembly may be outside or beyond the outer catheter 920. In some variations, the inner catheter 910 may be withdrawn from the outer catheter 920. For example, after the balloon assembly 930 has been used to deliver shockwaves to a patient's lumen, the balloon assembly 930 may be collapsed and the entire inner catheter 910 may be withdrawn. In such cases, the outer catheter 920 may be used to deliver pharmaceuticals or other instruments (tools, cameras, or the like) to the treatment region.

FIG. 9C shows a relationship between the inner catheter 910 and an outer catheter control shaft 921 when the catheter assembly 900 is in a second configuration. The outer catheter control shaft 921 may include a marking band 922. As described with respect to FIGS. 7A and 7C, the inner catheter 910 may include distal bands 911 and proximal bands 912. The distal bands 911 may be an example of the distal bands 711 of FIG. 7A and the proximal bands 912 may be an example of the proximal bands 712. The distal bands 911 and the proximal bands 912 may be used in conjunction with the marking band 922 to indicate relative position of the balloon assembly with respect to the outer catheter (not shown). In FIG. 9C, the marking band 922 is disposed between the proximal bands 912 indicating that the balloon assembly 930 is exposed or beyond the outer catheter 920. If the marking band 922 is disposed between the distal bands 911, then the balloon assembly 930 may be contained within the outer catheter 920.

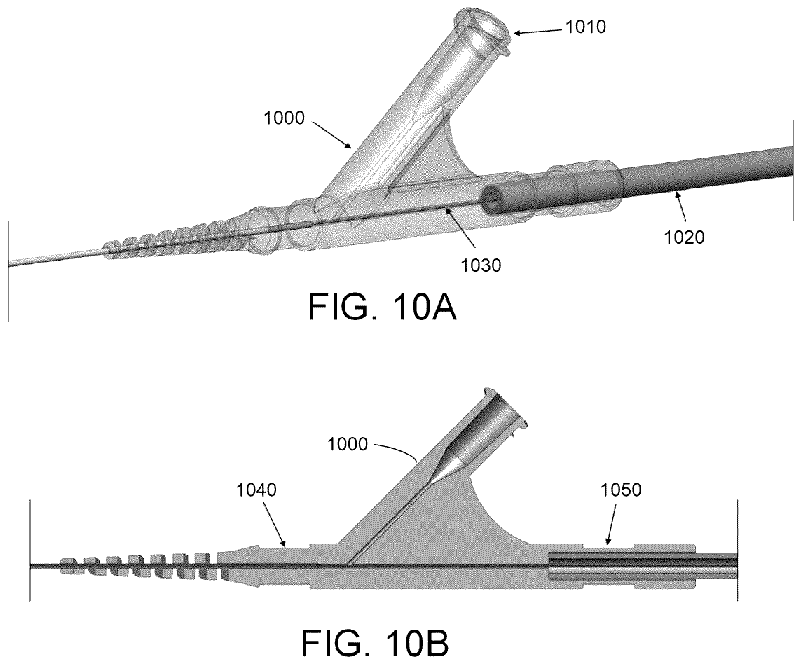

FIGS. 10A and 10B show various views of a hub 1000 that may be used in conjunction with the catheter assembly 300 of FIG. 3. In general, the hub 1000 may provide a control surface with which a clinician or other user can guide and/or control the catheter assembly 300. As shown in FIG. 10A, the hub 1000 may include an inflation port 1010. The inflation port 1010 can receive a liquid or gas to inflate a balloon assembly (not shown). In addition, a first end of a cable 1020 may be coupled to the hub 1000. A second end of the cable 1020 may be coupled to a generator (not shown), such as the generator 102 of FIG. 2. Within the cable 1020 can be one or more conductors 1030. The conductors 1030 may conduct a voltage, including a high voltage, from the generator 102 to one or more electrodes within the balloon assembly.

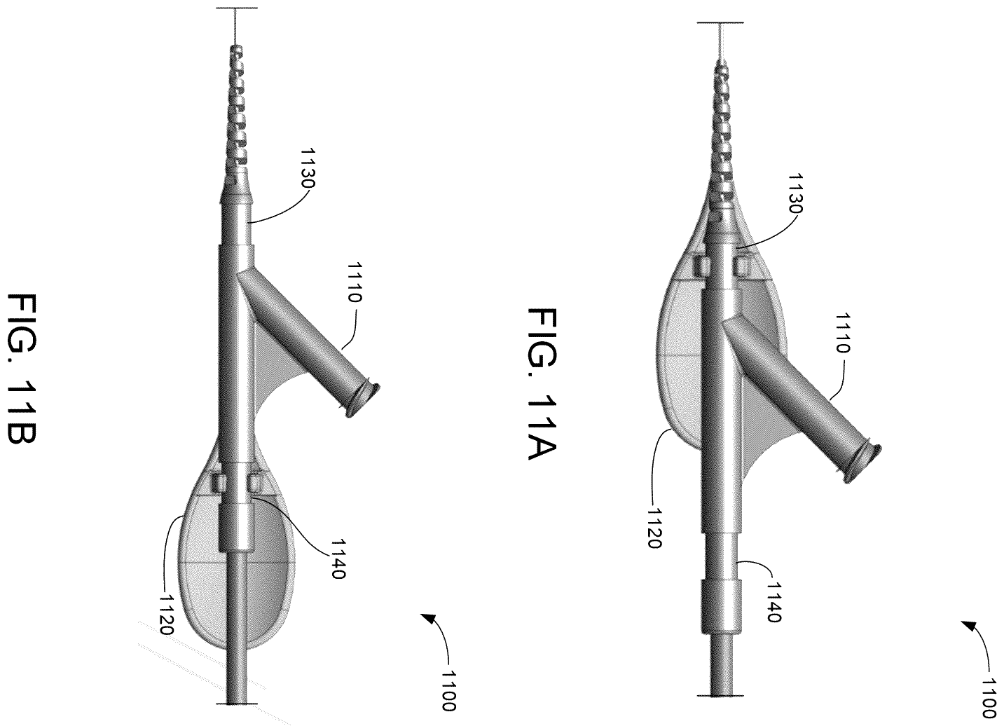

FIG. 10B shows a cross-sectional view of the hub 1000. As described above, the balloon assembly may be controlled by a positional relationship between the outer catheter control shaft and an inner catheter. In some examples, the inner catheter may be coupled to the hub 1000. The outer catheter control shaft may be external to the hub 1000. In some variations, a locking clip (not shown) may be coupled to the outer catheter control shaft. In turn, the locking clip may removably attach (e.g., clip) to the hub 1000 at either a first latch surface 1040 or a second latch surface 1050. This is described in more detail below in conjunction with FIGS. 11A and 11B.

FIGS. 11A and 11B show an example implementation of a latch mechanism 1100. FIG. 11A, in particular, shows the latch mechanism 1100 in a first configuration. In the first configuration, a balloon assembly (not shown) coupled to an inner catheter (also not shown) may be sheathed or enclosed by an outer catheter(not shown).

The latch mechanism 1100 may include a hub 1110 and a locking clip 1120. The hub 1110 may be an example of the hub 1000 of FIGS. 10A and 10B. The hub 1110 may include a first latch surface 1130 and a second latch surface 1140. The locking clip 1120 may be coupled to an outer catheter control shaft (not shown).

FIG. 11A shows the hub 1110 and the locking clip 1120 in a first configuration. For example, FIG. 11A shows the locking clip 1120 clipped (removably attached) to a first latch surface 1130. In the first configuration, the locking clip 1120 may position the outer catheter so that the balloon assembly (not shown) may be sheathed and enclosed by the outer catheter.

FIG. 11B shows the latch mechanism 1100 in a second configuration. The locking clip 1120 may be removably attached to the second latch surface 11140. In the second configuration, the locking clip 1120 may position the outer catheter so that the balloon assembly (not shown) may be un-sheathed and exposed from the outer catheter.

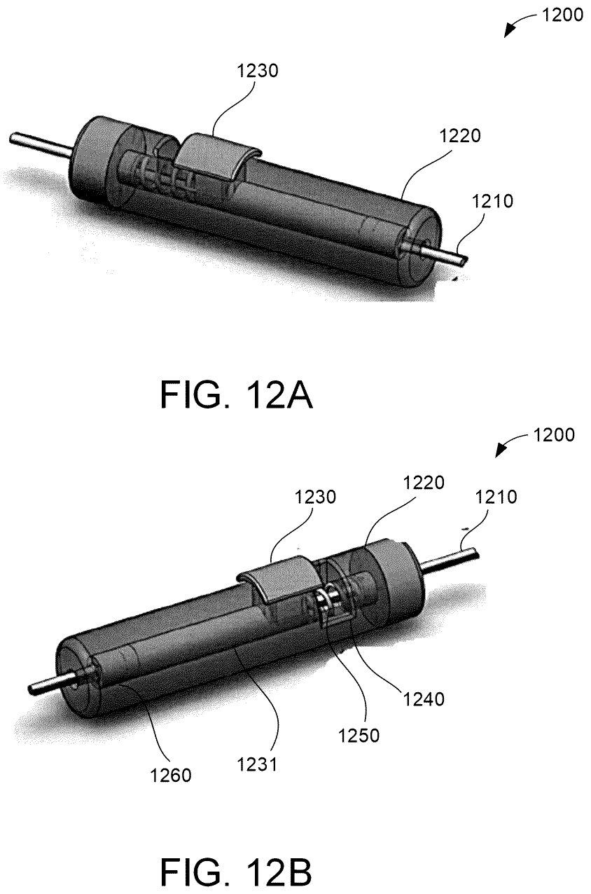

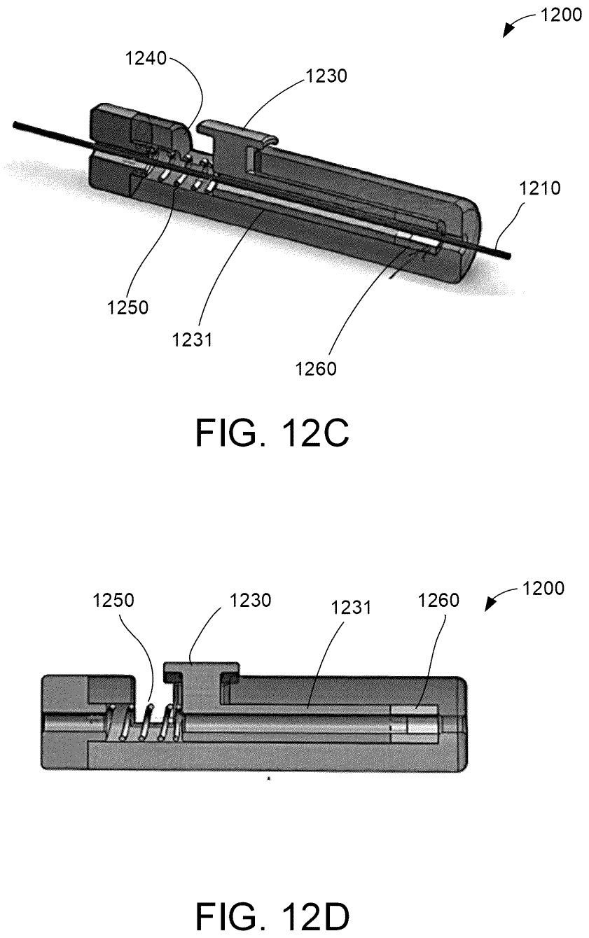

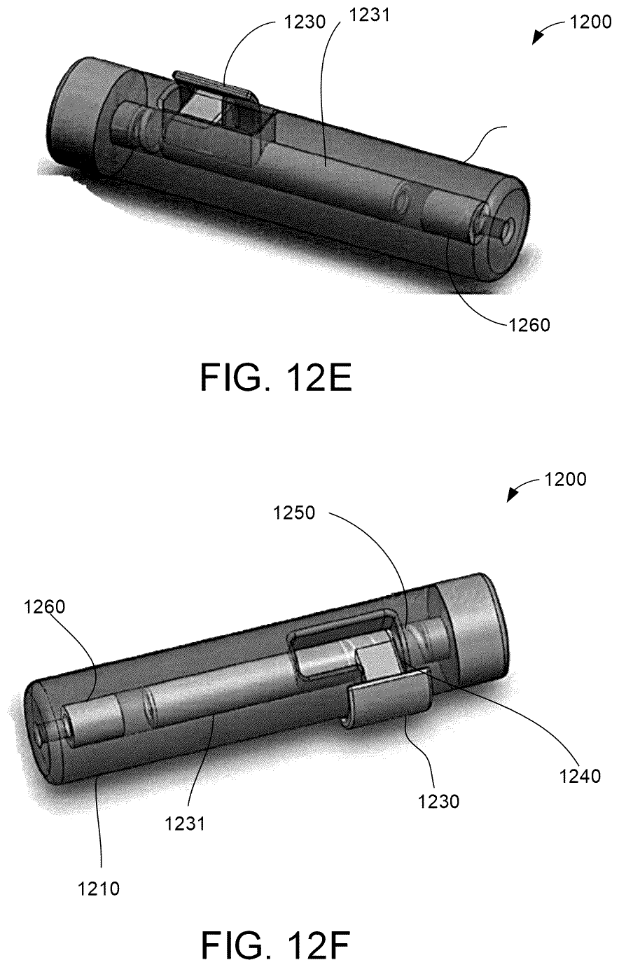

FIGS. 12A-12F show a first midshaft locking mechanism 1200. The midshaft locking mechanism 1200 may temporarily lock or bind together two (or more) catheters or shafts 1210. The shafts 1210 may include an outer catheter control shaft and an inner catheter as shown in FIG. 9C. In other examples, the shafts 1210 may include any feasible catheters or shafts that are desired to be locked together (e.g., made relatively immobile with respect to each other). In some embodiments, the midshaft locking mechanism 1200 may perform locking operations provided by the latch mechanism 1100 of FIGS. 11A and 11B.

FIG. 12A shows a perspective view of the midshaft locking mechanism 1200. The midshaft locking mechanism 1200 may include a cylindrical body 1220, and a button 1230. The shafts 1210 may be inserted through a central opening concentric to the body 1220.

FIG. 12B shows another perspective view of the midshaft locking mechanism 1200. FIG. 12B shows a slot 1240 for the button 1230 and a spring 1250 to provide a continuous force to bias the button 1230. Note that the button 1230 may be coupled to a plunger 1231 that applies force (such as a bias force) to a compliant (deformable) grommet 1260. The applied force may vary based on a position of the plunger 1231 and/or button 1230. The grommet 1260 may bind or lock together the shafts 1210 when deformed or otherwise compressed by the plunger 1231. Note that the shafts 1210 are passed through the grommet 1260 as well as an opening concentric with the body 1220. FIGS. 12A and 12B show the midshaft locking mechanism 1200 in a first (lock) mode. In the first mode, the midshaft locking mechanism 1200 locks together the shafts 1210. In some versions, the force from the spring 1250 is directly or indirectly used to lock together the shafts 1210. As will be described in FIGS. 12C and 12D, the slot 1240 may be used to place the midshaft locking mechanism 1200 in a second (unlock) mode.

FIG. 12C shows a cross-section view of the midshaft locking mechanism 1200 operated in the first mode. The spring 1250 provides a force to the button 1230 and plunger 1231, which in turn compresses and/or deforms a grommet 1260. Deformation of the grommet 1260 may provide a force that locks together the shafts 1210. Note that in the first mode, the button 1230 is not positioned into the slot 1240.

FIG. 12D shows another cross-section view of the midshaft locking mechanism 1200. FIG. 12D shows the spring 1250 pushing against the button 1230 and plunger 1231 which, in turn, pushes against the grommet 1260. The grommet 1260 may be any material (such as an elastomeric material) that can deform under pressure such as rubber, silicon, thermoplastic, or any other feasible material.

FIG. 12E shows a perspective view of the midshaft locking mechanism 1200 operated in a second (unlock) mode. In the unlock mode, any shafts (not shown) may freely move back and forth within the body 1220. Furthermore, in the unlock mode, the button 1230 and plunger 1231 may be moved away from the grommet 1260.

FIG. 12F shows another perspective view of the midshaft locking mechanism 1200 operated in the second mode. The button 1230 may be pressed against the spring 1250 and moved into the slot 1240. In this manner, the plunger 1231 may be held away from the grommet 1260, allowing the grommet to recover from deformation and allow any shafts (not shown) to move freely within the body 1220.

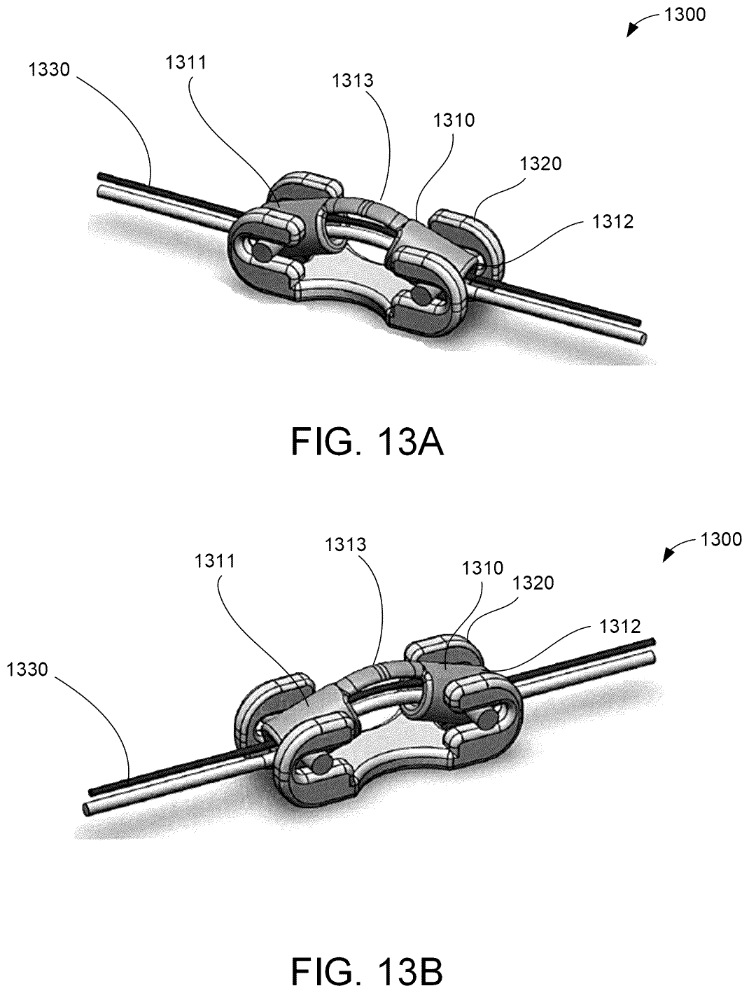

FIGS. 13A and 13B show a second midshaft locking mechanism 1300. The midshaft locking mechanism 1300 may include a clamp 1310 and a body 1320. Shafts 1330 may be passed through openings in the clamp 1310. As the shafts 1330 travel through the clamp 1310, the shafts 1330 may curve and arch. The clamp 1310 may include a first tube 1311, a second tube 1312, and a compliant arm 1313 configured to couple the first tube 1311 to the second tube 1312. In some examples, the arm 1313 may hold the first tube 1311 at an angle with respect to the second tube 1312. That is, the arm 1313 may cause the shafts 1330 to curve and arch as they pass between the first tube 1311 and the second tube 1312. The curve and arch of the shafts 1330 may cause the shafts to interfere and/or bind with the clamp 1310. In this manner, the clamp 1310 may bind the shafts 1330 together. The clamp 1310 can be made from any compliant material including nylon or any other elastomeric material. The material for the clamp 1310 may enable the arm 1313 to be depressed allowing the angle between the first tube 1311 and the second tube 1312 to be reduced. When the angle between the first tube 1311 and the second tube 1312 is reduced, then the shafts 1330 may not be bound together and may move (slide) freely within the midshaft locking mechanism 1300. The clamp 1310 may include locating pins that move within slots located within the body 1320, particularly with the arm 1313 is depressed.

FIG. 13B shows another perspective view of the midshaft locking mechanism 1300. As described with respect to FIG. 13A, to operate the midshaft locking mechanism 1300 in a second (unlock) mode, the user may deform the clamp 1310 by pressing down on the arm 1313 toward the body 1320 to at least partially straighten (align) the openings in the first tube 1311 and the second tube 1312. In this manner, the shafts 1330 may no longer curve, arch, or bind with the clamp 1310. Thus, in this mode the midshaft locking mechanism 1300 may slide onto virtually any position on the shafts 1330. Since the first tube 1311 and the second tube 1312 have limited openings, the shafts 1330 must be slid through the clamp 1310 prior to use. In general, the shafts 1330 are passed through the clamp 1310 during an initial assembly of a related catheter assembly.

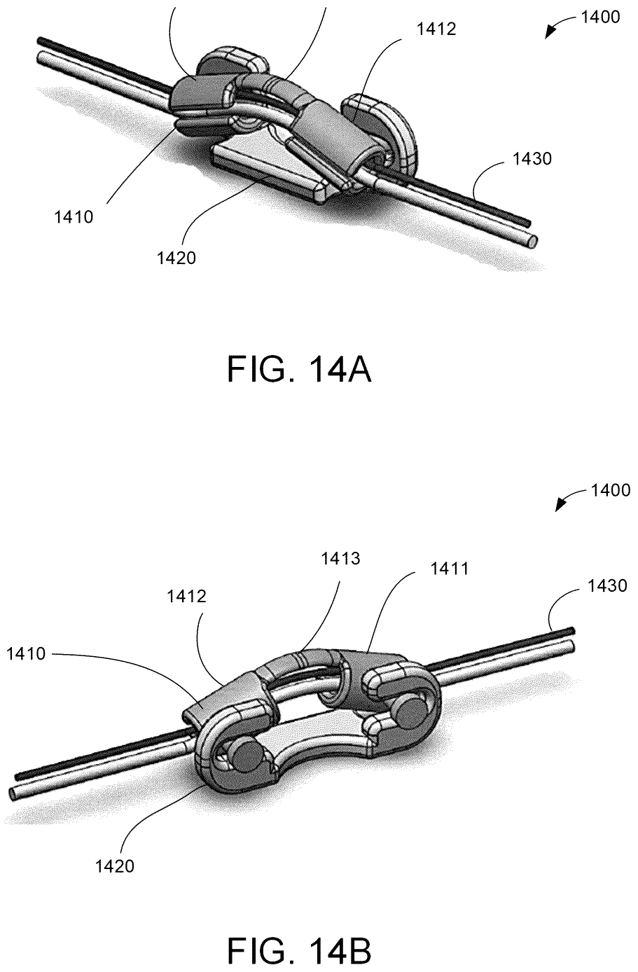

FIGS. 14A and 14B show a third midshaft locking mechanism 1400. Similar to the midshaft locking mechanism 1300 of FIGS. 13A and 13B, the midshaft locking mechanism 1400 may include a clamp 1410 and a body 1420. The clamp 1410 may include a first tube 1411, a second tube 1412, and an arm 1413. Similar to the midshaft locking mechanism 1300 of FIGS. 13A and 13B, the arm 1413 may cause shafts 1430 to curve and arch as they pass between the first tube 1411 and the second tube 1412. The curve of the shafts 1430 with respect to the clamp 1410 may cause the shafts 1430 to bind or lock together.

In some examples, the shafts 1430 may pass through slots in the first tube 1411 and the second tube 1412. Thus, the slots may enable the midshaft locking mechanism 1400 to be easily inserted or removed with respect to the shafts 1430 (particularly compared to the midshaft locking mechanism 1300 of FIGS. 13A and 13B.

FIG. 14B shows another perspective view of the midshaft locking mechanism 1400. As shown, the arm 1413 may be depressed toward the body 1420 to at least partially straighten (align) the openings in the first tube 1411 and the second tube 1412. In the shafts 1430 may no longer curve, arch, or bind with the clamp 1410.

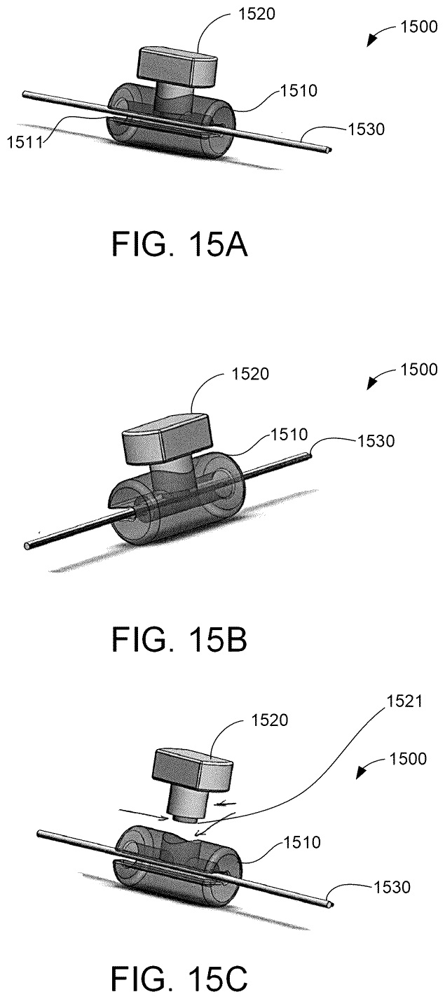

FIGS. 15A-15C show a fourth midshaft locking mechanism 1500. FIG. 15A shows a perspective view of the midshaft locking mechanism 1500 that may include a body 1510 and a cam 1520. The body 1510 may include a slot 1511 to receive two or more shafts 1530.

The cam 1520 may contact and pinch the shafts 1530 toward the body 1510 in order to bind the shafts 1530 to the body 1510. In some variations, the cam 1520 may be designed to turn one-fourth of a whole turn (e.g., a quarter-turn). Thus, a user can turn the cam 1520 a quarter turn to bind or release the shafts 1530 from the body 1510. A first position of the cam 1520 may allow the shafts 1530 to move freely (with respect to the body 1510. A second position of the cam 1520 may bind the shafts 1530 to the body 1510 locking the shafts 1530 together. FIG. 15B shows another perspective view of the midshaft locking mechanism 1500. In this view, the body 1510, the cam 1520, and the shafts 1530 are visible while the slot 1511 is occluded by the body 1510.

FIG. 15C shows an exploded view of the midshaft locking mechanism 1500. The midshaft locking mechanism may include a body 1510 and a cam 1520. The cam 1520 may include an elastomeric stopper 1521 that can contact the shafts 1530 when the cam 1520 is turned. The elastomeric stopper 1521 can both protect the shafts 1530 from damage and also increase a binding force applied by the cam 1520. The elastomeric stopper 1521 can be rubber, silicon, thermoplastic urethane, or other feasible material.

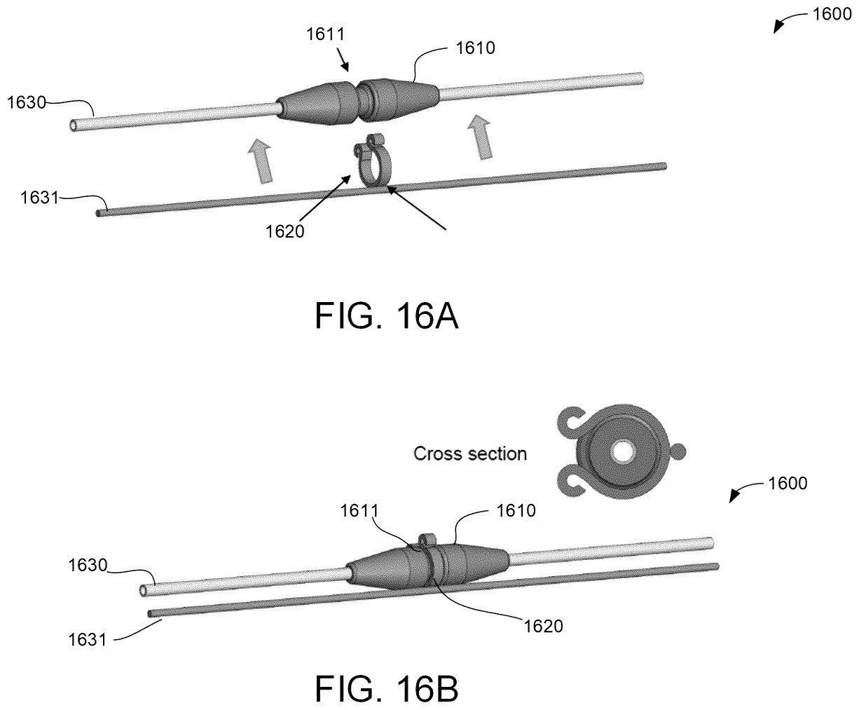

FIGS. 16A and 16B show a fifth midshaft locking mechanism 1600. The midshaft locking mechanism 1600 can lock together to shafts or catheters. The midshaft locking mechanism 1600 may include a locking ring 1610 and a locking clip 1620. The locking ring 1610 is shown mounted to a first shaft 1630 and the locking clip 1620 is shown attached to a second shaft 1631. In some examples, the locking clip 1620 may be formed from nitinol and may be welded to the second shaft 1631.

The locking ring 1610 may be formed from any material that may be molded or machined. The locking ring 1610 may include a groove 1611 that is designed to receive the locking clip 1620. To lock the first shaft 1630 to the second shaft 1631, the locking clip is snapped into the groove 1611 of the locking ring 1610.

FIG. 16B shows the midshaft locking mechanism 1600 when the first shaft 1630 is locked to the second shaft 1631. As shown, the locking clip 1620 is clipped into the groove 1611 of the locking ring 1610.

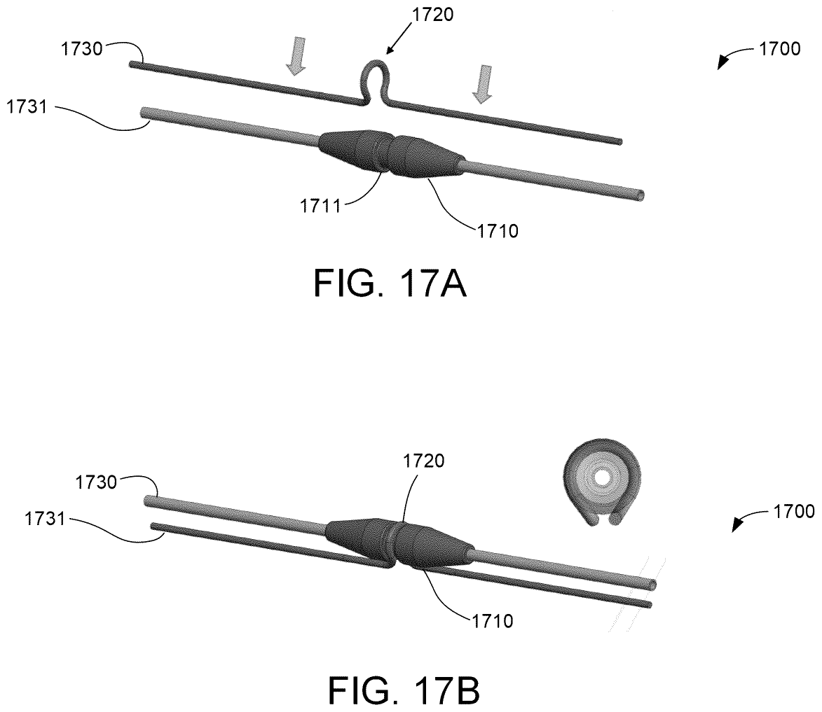

FIGS. 17A and 17B show a sixth midshaft locking mechanism 1700. The midshaft locking mechanism 1700 can lock together a first shaft 1730 and a second shaft 1731. The midshaft locking mechanism 1700 can include a locking ring 1710 and a locking loop 1720. In some variations, the locking loop 1720 may be formed with the first shaft 1730. For example, the first shaft 1730 may be formed from nitinol or other similar material.

The locking ring 1710 may be another example of the locking ring 1610 of FIGS. 16A and 16B. Thus, the locking ring 1710 may include a groove 1711 designed to receive the locking loop 1720.

FIG. 17B shows the midshaft locking mechanism 1700 when the first shaft 1730 is locked to the second shaft 1731. As shown, the locking loop 1720 is clipped into the groove 1711 of the locking ring 1710.

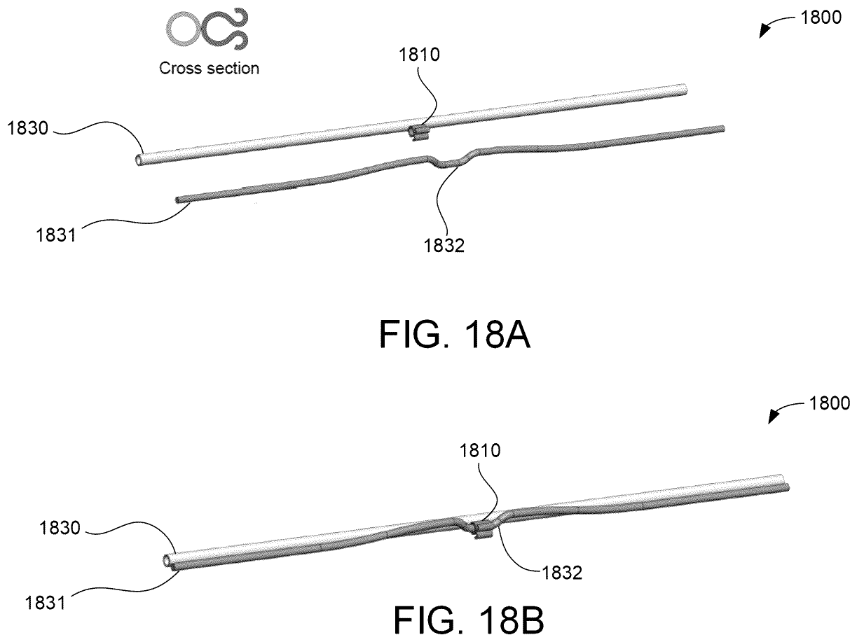

FIGS. 18A and 18B show a seventh midshaft locking mechanism 1800. The midshaft locking mechanism 1800 can lock together a first shaft (or catheter) 1830 and a second shaft (or catheter) 1831. The midshaft locking mechanism 1800 may include a snap ring 1810 that is attached to at least one of the shafts. In the example midshaft locking mechanism 1800, the snap ring 1810 is attached to the first shaft 1830, however in other examples, the snap ring 1810 may be attached to any feasible shaft.

The second shaft 1831 may include a notch 1832 that is bent into the second shaft 1831. The notch 1832 may be configured to snap into the snap ring 1810. In some examples, the snap ring 1810 may be a metal or metallic material such as nitinol or the like. In other examples, the snap ring 1810 may be an elastomeric material. The snap ring 1810 may be attached to the first shaft 1830 by welding, adhesive, or other feasible means. FIG. 18B shows the midshaft locking mechanism 1800 when the first shaft 1830 is locked to the second shaft 1831. As shown, the notch 1832 is clipped into the snap ring 1810.

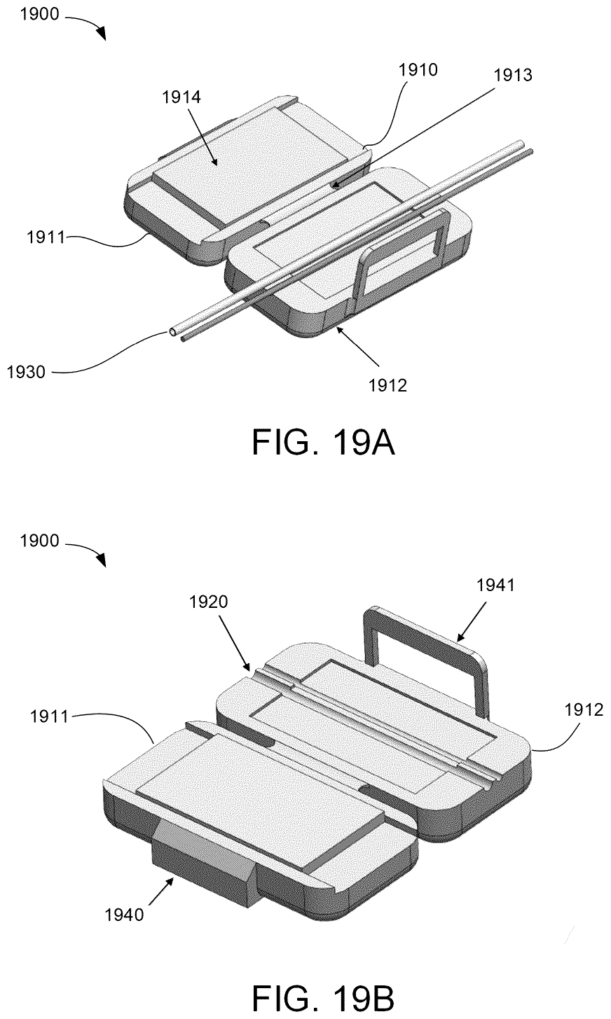

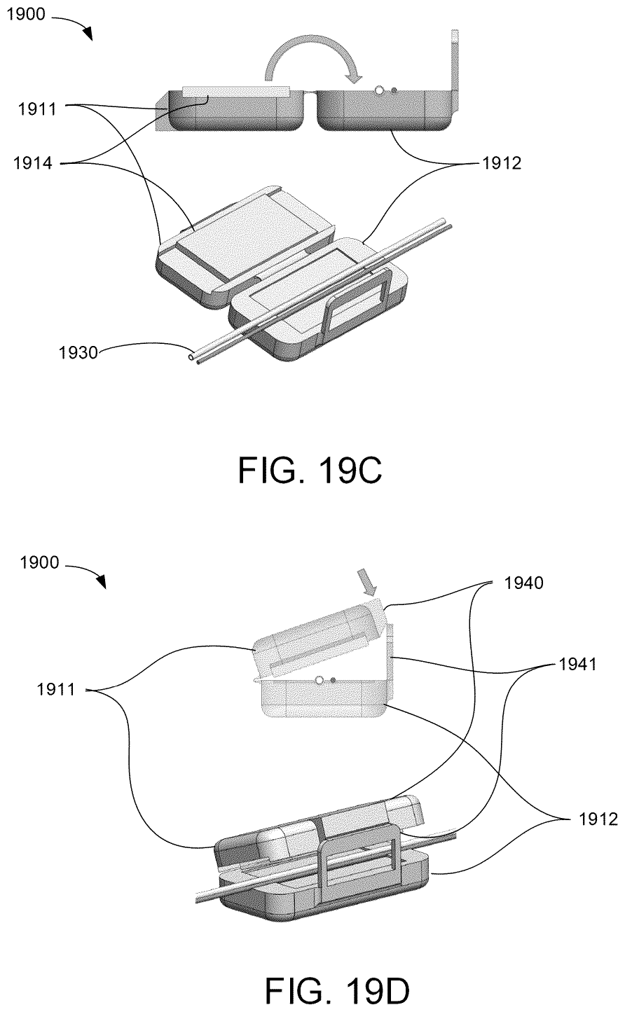

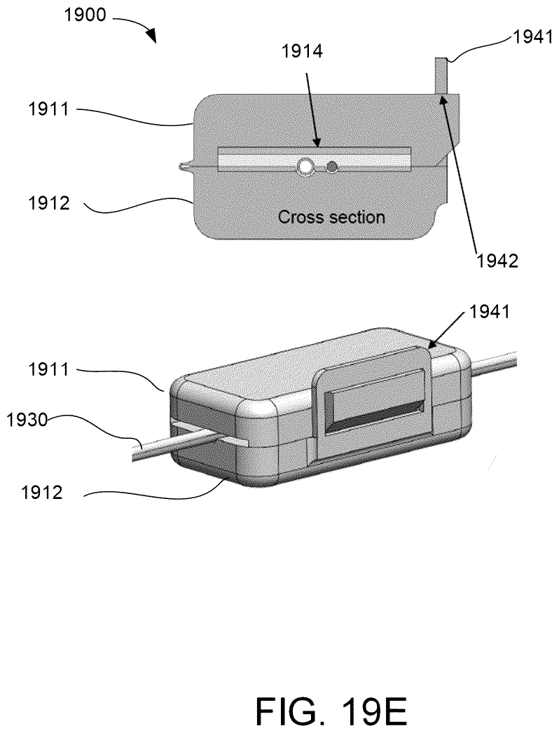

FIGS. 19A-19E show an eighth midshaft locking mechanism 1900. FIG. 19A shows the midshaft locking mechanism 1900 open and receiving two or more shafts 1930. The midshaft locking mechanism 1900 can lock together the shafts (or catheters) 1930. The midshaft locking mechanism 1900 can include a clam shell body 1910. The clam shell body 1910 may include a first member 1911, a second member 1912, a hinge member 1913, and an elastomeric pad 1914.

The first member 1911 can be movably coupled to the second member 1912 through the hinge member 1913. The clam shell body 1910 can be molded, including injection molded. Thus, the hinge member 1913 can possess the material properties (bendability, flexibility, etc.) of the clam shell body 1910.

As shown, the elastomeric pad 1914 may be attached to the first member 1911. In other examples, the elastomeric pad 1914 may be attached to the second member 1912. The elastomeric pad 1914 can lock or bind together the shafts 1930 by applying pressure and/or friction to the shafts 1930 and the clam shell body 1910. The elastomeric pad 1914 can be thermoplastic urethane, silicon, rubber, or the like.

FIG. 19B shows a perspective view of the midshaft locking mechanism 1900. The first member 1911 includes the elastomeric pad 1914 and the second member 1912 includes two or more grooves 1920 to receive and/or guide the shafts 1930. In addition, the clam shell body 1910 may include a latch mechanism to latch the first member 1911 to the second member 1912. As shown, the first member 1911 can include a ramp member 1940 and the second member 1912 can include a beam member 1941. The beam member 1941 can interact with the ramp member 1940 to hold the first member 1911 to the second member 1912 such that the elastomeric pad 1914 is in contact with the shafts 1930. The ramp member 1940 may be molded onto an edge of the first member disposed across from the hinge member 1913. The beam member 1941 may be molded onto an edge of the second member 1912, the edge of the second member 1912 disposed across from the hinge member 1913, where the beam member 1941 is configured to engage and detachably couple to the ramp member 1940.

FIG. 19C shows other views of the midshaft locking mechanism 1900. The shafts 1930 are disposed within the second member 1912. In this example, the first member 1911, which includes the elastomeric pad 1914 may be closed onto the second member 1912 to lock or bind the shafts 1930 to each other.

FIG. 19D shows the midshaft locking mechanism 1900 as the first member 1911 begins to close and latch to the second member 1912. As shown, the beam member 1941 interacts with the ramp member 1940. In some cases, the ramp member 1940 can deflect or move the beam member 1941.

FIG. 19E shows the midshaft locking mechanism 1900 in a locked configuration. The beam member 1941 may be in contact with a locking surface 1942 of the ramp member 1940. In this configuration, the first member 1911 may be held in contact with the second member 1912 by the beam member 1941. Thus, the elastomeric pad 1914 may contact and bind the shafts 1930 to the second member 1912. To open the midshaft locking mechanism 1900, a user would move the beam member 1941 off of the locking surface 1942.

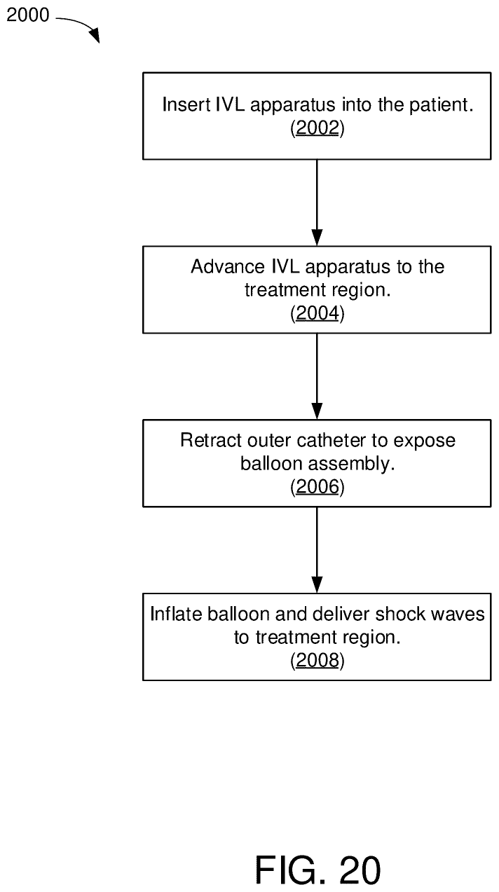

FIG. 20 is a flowchart showing an example method 2000 for treating a patient with an IVL system. Some examples may perform the operations described herein with additional operations, fewer operations, operations in a different order, operations in parallel, and some operations differently. The method 2000 is described below with respect to the IVL system 100 of FIG. 1, however, the method 2000 may be performed by any other suitable system or device.

The method 2000 begins in block 2002 as an IVL apparatus is inserted into a patient. The IVL apparatus may include a catheter assembly 130 and/or an balloon assembly 140. In some applications, the IVL apparatus is inserted percutaneously into the patient vein, artery, or other lumen.

Next, in block 2004, the IVL apparatus is advanced into a treatment region. That is, the IVL apparatus is moved into position, and in some cases the position of the IVL apparatus may be verified using feasible means (e.g., x-rays, sonogram, etc.). In some cases, the radiopaque portions of the catheter assembly 130 and/or the balloon assembly 140 may help determine the position of some or all of the IVL system 100. In some examples, advancing the IVL apparatus may include locking two or more catheters or shafts together. For example, an outer catheter may be locked together with an inner catheter.

Next, in block 2006, the outer catheter of the catheter assembly 130 may be retracted to expose the balloon assembly 140. The relationship between the outer catheter and the balloon assembly is described in detail in conjunction with FIGS. 4-9. In some examples, retracting the outer catheter may include unlocking two or more catheters or shafts. In some other examples, retracting the outer catheter may include viewing or identifying a marker band or ring within an alignment window disposed on an outer catheter.

Next, in block 2008, the balloon included in the balloon assembly 140 is expanded or inflated and shockwaves are delivered to the treatment region. For example, a gas or liquid may be introduced into the balloon assembly 140 to expand the balloon. Following the expansion, high voltage pulses may be introduced to electrodes to generate shockwaves.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An intravascular lithotripsy apparatus comprising:
an inner catheter comprising;
a tapered element coupled to a distal end of the inner catheter; and
a balloon assembly coupled to the inner catheter disposed proximal to the tapered element, wherein the balloon assembly includes one or more electrode pairs configured to emit an electric arc; and
an outer catheter comprising:
a lumen configured to concentrically surround and slidably couple to the inner catheter; and
a tip disposed on a distal end of the lumen and configured to slidably engage with a landing zone of the tapered element.

2. The intravascular lithotripsy apparatus of claim 1, wherein the balloon assembly is configured to be inflated by at least one of a liquid or a gas.

3. The intravascular lithotripsy apparatus of claim 1, wherein the balloon assembly is configured to be contained within the outer catheter when the tip of the outer catheter is in contact with the landing zone of the tapered element.

4. The intravascular lithotripsy apparatus of claim 1, wherein the outer catheter further comprises an alignment window and the inner catheter further comprises a midshaft marker configured to appear within the alignment window when the tip of the outer catheter is in contact with the landing zone of the tapered element.

5. The intravascular lithotripsy apparatus of claim 1, further comprising two or more markers configured to indicate a position of the balloon assembly with respect to the outer catheter when the intravascular lithotripsy apparatus is inserted into a patient.

6. The intravascular lithotripsy apparatus of claim 5, wherein the two or more markers remain outside the patient.

7. The intravascular lithotripsy apparatus of claim 1, further comprising:
a control shaft coupled to a proximal end of the outer catheter.

8. The intravascular lithotripsy apparatus of claim 7, wherein the inner catheter comprises a first set of marker bands configured to indicate when the balloon assembly is within the outer catheter.

9. The intravascular lithotripsy apparatus of claim 7, wherein the inner catheter comprises a second set of marker bands configured to indicate when the balloon assembly is extended beyond the outer catheter.

10. The intravascular lithotripsy apparatus of claim 7, further comprising:
a hub coupled to the inner catheter; and
a locking clip coupled to the control shaft, wherein the locking clip is configured to lock the balloon assembly in a first configuration where the balloon assembly is outside of the outer catheter and in a second configuration where the balloon assembly is inside of the outer catheter.

11. The intravascular lithotripsy apparatus of claim 1, wherein the two or more electrodes are formed from a flex circuit, conductive ink, or a combination thereof.

12. The intravascular lithotripsy apparatus of claim 1, wherein the tapered element is about three centimeters long.

13. The intravascular lithotripsy apparatus of claim 1, wherein the tip of the outer catheter and the tapered element form a smooth outer surface when the tip of the outer catheter is slidably engaged with the landing zone of the tapered element.

14. The intravascular lithotripsy apparatus of claim 1, wherein the inner catheter is configured to be withdrawn from the outer catheter.

15. The intravascular lithotripsy apparatus of claim 1, wherein the balloon assembly is between two and eight millimeters in diameter when the balloon assembly is inflated.

16. The intravascular lithotripsy apparatus of claim 1, wherein the balloon assembly is at least 20 millimeters long.

17.-21. (canceled)
